(19) United States
(12) Patent Application Publication
(10) Pub. No.: US 2007/0055166 A1
(43) Pub. Date: Mar. 8, 2007

(54) Method and system for recording and transmitting data from biometric sensors

(76) Inventor: Chandrashekhar Patil, Oceanside, CA (US)

Correspondence Address: G. L. LOOMIS & ASSOCIATES, INC., 990 HIGHLAND DRIVE, SUITE 212Q, SOLANA BEACH, CA, 92075, US

(21) Appl. No.: 11/219,197

(22) Filed: Sep. 2, 2005

(51) Int. Cl. A61B 5/04; A61B 5/00; A61B 10/00

(52) U.S. Cl. 600/509; 600/300; 600/549; 600/565; 128/903

(57) ABSTRACT

Telemedical systems that allow physiological signals from biometric sensors affixed to a medical patient to be monitored at remote sites. These systems afford a method for transmitting analog signals from the biometric sensors to a self-contained module wherein the signal is conditioned, digitized, stored and transmitted to a local host that in turn transmits the data to a remote host. Overall, the telemedical systems herein described allow for transmission of data to one or more remote locations in real time from sensors affixed to a patient. Additionally, these telemedical systems allow for the data obtained from the biometric sensors to be stored within in the module for later transmission.

### FIELD OF THE INVENTION

This invention belongs to the field of medical electronics and relates to systems for acquisition and telemetry of medical diagnostic data for remote patient monitoring. More specifically, the invention relates to methods and systems utilizing a self-contained module for the recording, conditioning and transmitting of medical data from biometric sensors affixed to a patients body for diagnostic purposes.

### BACKGROUND OF RELATED ART

Systems for mobile diagnostic monitoring of medical patients are know in the art and several of these systems allow the physiologic data of patients within a medical facility to be monitored remotely using wireless communications. Such systems often include remote transmitters or transceivers that collect and transmit physiologic data from respective patients over a wireless channel. This physiologic data may include, for example, real-time electrocardiograph (EKG) waveforms, blood oxygen levels, and non-invasive blood pressure readings. A system for mobile EKG monitoring of high-risk patients is described in U.S. Pat. No. 6,535,758 and a method and system for hand-held medical monitoring is disclosed in U.S. Pat. No. 6,654,631.

Some systems include battery-powered remote transceiver devices that are adapted to be worn by or attached to patients allowing such patients to be monitored while ambulatory. Several types of recorders that are worn on the body have also become known. So-called long-term EKG recorders generally record the EKG of the patient continuously, usually over a period of 24 hours, by means of an electronic memory. Subsequently the data is read out or removed and may be evaluated at some time later.

Recently, recorders with internal memory devices that can be implanted into the body have become known. Such implantable recorders with internal EKG memory devices allow data to be stored for up to approximately one hour and the EKG data can be read out externally. However, implanted recorders must then be replaced after about 1 year. Furthermore, the implantation of a device for diagnostic purposes is generally only suitable for a small number of very high-risk patients.

There are several deficiencies of the current state of the art. One shortfall stems from a lack of standardization of the communication devices used for data transmission and points to a need for more universal system compatibility.

For example, U.S. Pat. No. 6,654,631 to Sahai describes a system that utilizes a hand-held device such as a personal digital assistant (PDA) to interface directly with a patient and wherein the signal is digitized directly in the PDA. This arrangement compromises patient safety since such PDA devices are not designed to the rigorous international standards required for medical device safety. Furthermore, this system as described is prone to loss of data in the event of an interruption of the electronic connection to a host or remote device. U.S. Pat. No. 6,535,758 to Sharner et al describes a method for recording and transmitting multi-channel ECG signals directly to a mobile phone via a hard-wired serial interface. Similarly, this arrangement also compromises patient safety. Furthermore, neither these systems address the need for electrical isolation that is also necessary for patient safety.

Therefore, a need exists for systems that utilize a standardized set of devices in order to minimize manufacturing costs, user training and service to the system. A need also exists for a system that prevents loss of data in the event of an interruption of an electronic connection to a host or remote device. Furthermore, a need exists for a system in which electrically wired connections are electrically isolated. The present invention addresses these and other problems associated with devices presently known in the art by providing several inventive features that may be used individually or in appropriate combination.

### BRIEF DESCRIPTION OF THE DRAWINGS

FIG. 1 is block diagram that schematically illustrates the invention.

FIG. 2 is a schematic diagram of a wireless ECG device affixed to a body

### SUMMARY OF THE INVENTION

For the purposes of the present invention the terms EKG and ECG are used synonymously to mean electrocardiography or electrocardiogram; the term A/D converter means is used to mean any device used to convert an analog signal to a digital signal; and the term USB is used to mean a Universal Serial Bus.

The present invention describes telemedical systems that allow physiological signals from biometric sensors affixed to a medical patient to be monitored at remote sites. These systems afford a method for transmitting analog signals from the biometric sensors to a self-contained module wherein the signal is conditioned, digitized, stored and transmitted to a local host that in turn transmits the data to a remote host. Overall, the telemedical systems herein described allow for transmission of data to one or more remote locations in real time from sensors affixed to a patient. Additionally, these telemedical systems allow for the data obtained from the biometric sensors to be stored within in the module for later transmission.

The biometric sensors useful in the present invention include, but are not limited to multi-channel electrocardiograms (EKG) such as 12-lead electrocardiograms and 3-lead electrocardiograms, blood oxygen sensors, pulmonary sound sensors, pulmonary function sensors, temperature sensors, blood pressure sensors, blood glucose level sensors, and patient image sensors.

An important feature of the systems of the present invention is the use of electrically isolated devices that permit both high-speed wire and wireless connection to the local host. Subsequent transmission of the data to the remote host is achieved via communication systems such as a cellular phone network or a public Internet.

Another important feature of the systems herein described is the prevention of the loss of data in the event of an interruption of an electronic connection to a host or remote device by providing high capacity local memory means for data storage. This arrangement also allows for either continuous monitoring over a period of days or periodic monitoring over periods of weeks or months. In addition, the use of removable storage devices permits continuous or periodic monitoring of patients nearly indefinitely. Furthermore, to insure data security these systems may employ secure encrypted links to the remote host.

### DETAILED DESCRIPTION OF THE INVENTION

The systems herein described afford methods for the recording and transmittance of electrical signals from biometric sensors and monitors affixed to the body of a patient for diagnostic purposes to be monitored from remote locations. The key component of the system is a self-contained module in electronic communication with one or more of these biometric sensors. The self-contained module is capable of simultaneous real time transmission and on-board storage of patient data. This unique and important feature of the present invention assures that no data is lost in the event of a disruption of the real time transmission to the local host. In turn the local host is capable of transmitting the data to a remote host in real time or of storing the data in on-board memory for later transmission.

These systems find use in the following situations:
1. Allow Emergency Medical Response (EMR) staff to monitor and record physiological data of a patient for the duration of a journey to a health care facility wherein data can be transferred to a host computer for a medical practitioner to review the patient's immediate physiological history.
2. A medical facility after discharge of a cardiovascular patient, can remain in contact with the patient. The patient is provided with a multiple lead EKG terminal spread placed on the body, and the signals are collected and transmitted to a remote central location. At the central location, the transmitted EKG data is analyzed. It is compared with normal EKG signals and signals captured in time from the same patient as part of the patient history.
3. Telemedicine: Providing medical care to a population removed from standard hospital and physician services. Included are rural health initiatives in areas of the world with little or no medical care access.
4. Home health monitoring for patients who participate in plans to maintain health and monitor chronic conditions.
5. Nursing home care wherein patients can be monitored and diagnosed by remote medical facilities.
6. Accrual of comprehensive electronic medical records that allow the storage and exchange physiological data.

An important feature of the present invention is the utilization of a uniform set of devices to minimize manufacturing costs and the training needed to use and service the system. To this end, an aim of the present invention is to implement as local hosts universal and commercially available devices such as mobile telephones and personal digital assistants (PDA) allowing for portability and ease of use. However, such device are utilized in the systems of the present invention are utilized in a manner such that patient safety is not compromised.

In the following description, various aspects of the present invention are described. However, it will be apparent to those skilled in the art that the present invention may be practiced with either some or all aspects of the present invention. For purposes of explanation, specific numbers, materials and configurations are set forth in order to provide a thorough understanding of the present invention. However, it will also be apparent to one skilled in the art that the present invention may be practiced without the specific details. In other instances, some well-known features are omitted or simplified in order not to obscure the present invention.

Parts of the description will be presented in terms of operations performed by a computing device, using terms such as sensing, converting, comparing, storing, generating and so forth. As well understood by those skilled in the art, these quantities and operations take the form of electrical, magnetic, or optical signals capable of being stored, transferred, combined, and otherwise manipulated through mechanical and electrical components of a digital system. The term digital system includes general purpose as well as special purpose computing machines, systems, and the like, that are standalone, adjunct or embedded.

Various operations are described in a manner that is most helpful in understanding the present invention, however, the order of description should not be construed as to imply that these operations are necessarily order dependent.

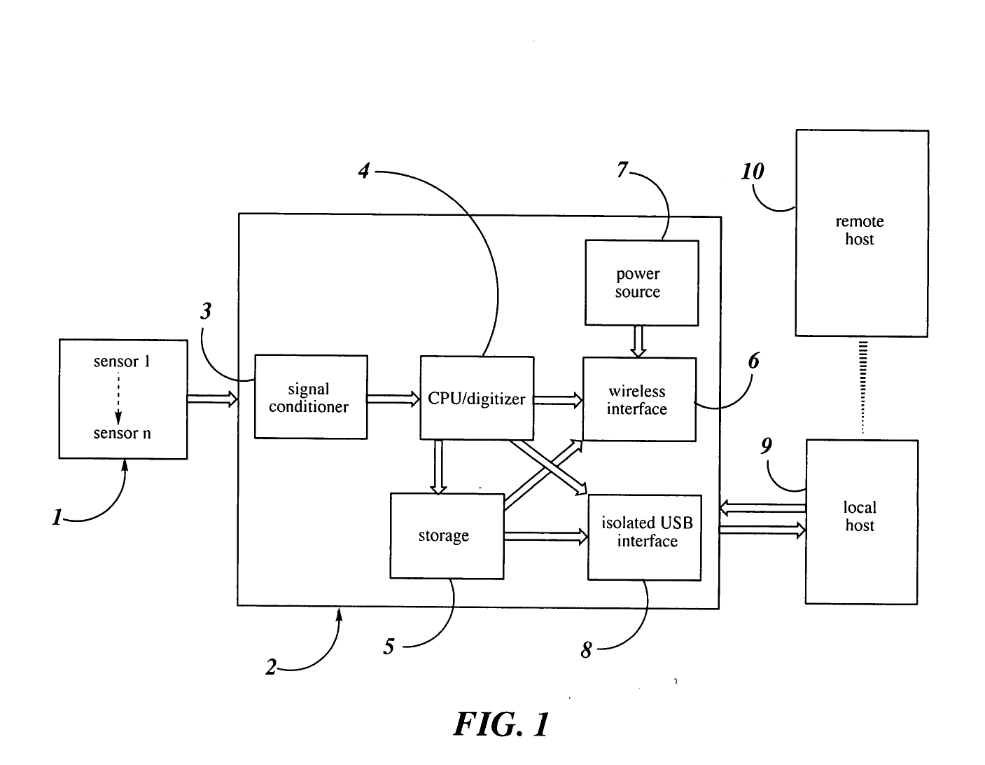

The following is a description of an embodiment of the telemetry systems of the present invention. With reference to FIG. 1, the present invention describes systems with the following principal elements: one or more biometric sensors 1 capable of being affixed to a patient and being electronically connected to module 2 via a first electronic communication link; wherein module 2 comprises a signal conditioner 3, an analog to digital (A/D) converter 4, a data storage means 5, a wireless interface 6 powered by power source 7, and/or an isolated Universal Serial Bus (USB) interface 8; and wherein the module 2 is capable of establishing and maintaining a second electronic communication link with a local host 9, said second electronic communication link being a wireless communication link utilizing wireless interface 6 or a high-speed wire communication link utilizing the isolated Universal Serial Bus (USB) interface 8. Subsequently the local host 9 is capable of establishing and maintaining a third electronic communication link with the remote host 10 for the transmission of data.

An embodiment of a method for remote patient monitoring utilizing the system described in FIG. 1 consists essentially of providing one or more biometric sensors 1 affixed to a patient and connected to the module 2 via a first electronic communication link; transmitting electronic signals from the biometric sensors 1 to module 2 via the first electronic communication link; converting, within the module 2, the electronic signals obtained from the biosensors 1 into digital data; transmitting the digital data from module 2 to local host 9 via a second electronic communication link; and finally transmitting said digital data from said local host to said remote host via said third electronic communication link.

Another embodiment of a method for remote patient monitoring utilizing the system described in FIG. 1 includes the step of storing the digital data utilizing digital data storage means 5 within module 2 while simultaneously transmitting the digital data to local host 9 via the second electronic communication link. This embodiment is realized by utilizing a data storage means 5 capable of storing biological data while simultaneously transmitting these data to the host. Exemplary of such data storage means are the Multi Media Card (MMC) and the DATAFLASH™ (available from Atmel Inc.).

In yet another embodiment of the present invention a method for remote patient monitoring utilizes the system described in FIG. 1 and includes the step of storing the digital data utilizing digital data storage means 5 within module 2 prior to the step of transmitting the digital data to local host 9 via the second electronic communication link.

A list of biometric sensors 1 suitable for use in the present invention includes but is not limited to 12-lead electrocardiograms (EKG), 3-lead electrocardiograms (EKG), blood oxygen sensors, pulse oximeters, pulmonary sound sensors, stethoscopes, pulmonary function sensors, temperature sensors, blood pressure sensors, blood glucose level sensors also know as glucometers, laryngoscopes and various patient image sensors including ultrasound and fetal monitors. Furthermore, the systems of the present invention can function as add-on devices to other more complex imaging devices such as portable X-ray, cardiac imaging devices, ultrasonic imaging, and radiology devices thus enabling these more complex imaging devices to be used more effectively in remote locations.

In addition to biosensors that transmit raw analog data, biosensors useful in the present invention may be of the type that are capable of digitizing and analyzing the biological data within the biosensor itself and transmitting a digitized and analyzed signal to the module 2. Such sensors are presently available for analyzing blood oxygen saturation and biosensors capable of digitizing and analyzing biological data are being developed for blood glucose meters, portable cholesterol measurement units and portable DNA diagnostic units.

The biosensors of the present invention may be affixed to the body of a patient by any known means. For example, certain biosensors that require intimate contact with the exterior of a particular area of the body may be held in place by various types of mechanical clips, flexible belts, elastic wraps, dermal adhesives, and the like. Other biosensors may be affixed to a patient via placement into body orifices, i.e. the mouth, nose, ear, vagina, urethra or anus with or without auxiliary fixation means.

The communication link between the biometric sensors 1 and the module 2 may be a wired connection or a wireless connection. The choice of such connection is may be dictated by the specific type of biometric sensors used and requirements for a specific application. For example, total mobility of the patient while recording and transmitting data such as EKG data can be ensured by utilizing wireless transmission of the signal from the biosensors to the module.

Signal conditioners 3 suitable for use in the present invention include amplifiers, multi-channel amplifiers, filters, amplifier/filter combinations, source encoders and other such similar devices. Non-limiting examples of such devices include software-implemented 50 Hz/60 Hz notch filters, low pass Butterworth filters, and low lag moving average filters and low pass filters implemented using operational amplifiers.

Devices particularly suitable to function as A/D converter 4 include but are not limited to the C8051Fxxx series of controllers, with integrated A/D and D/A (available from Silicon Labs, Austin Tex.) and HCS08 and HCS12 microcontrollers with integrated A/D and D/A (available from Freescale Semiconductors Inc, Irvine, Calif.).

Various on-board data storage means 5 suitable for use in module 2 may include any of the various types of random access memory (RAM), removable hard drives, drives with removable media, memory chips, flash-ROM, removable flash memory cards and other such devices either individually or in combination. Presently, such data storage devices have capacities in the range of about 50 to 1000 MB. By way of example, a data storage device with a capacity of 1000 MB is sufficient to store a patient's EKG data for about one month. The maximum capacity of the data storage device selected is a function of the specific sensor necessary for a specific application.

Suitable power sources for the wireless interface include any of the various types of storage batteries and similar devices such as the Li Ion Cell Charger MAX 1874 (available from Maxim Integrated Products, Sunnyvale, Calif.).

Local host 9 can be any device capable of receiving the data from the module 2 and transmitted the data to remote host 10. Devices suitable to function as local host 9 include personal digital assistants (PDA); various types of smart cell phones; various types of personal computers including pen computers and portable notebook computers. The remote host 10 can be any device capable of receiving the data from the local host 9. Suitable remote host 10 devices include personal digital assistants (PDA); various types of smart cell phones; various types of personal computers including pen computers and portable notebook computers; and various types of Internet server computers

The systems generally utilize electrically isolated devices that permit both high-speed wire and wireless connection to a local host. The USB interface may be the Master/Slave type or the slave only type. A preferred USB interface for use in the present invention is the CP210x UART-USB bridge (available from Silicon Labs, Austin Tex.).

Preferred devices for wireless transmission to the local host include the ROK104001 multichip module (available from Infineon Technologies AG, Munchen, Germany); the 13192 Zigbee module (available from Freescale Semiconductors Inc, Irvine, Calif.); and the LMX9820A Bluetooth module (available from National Semiconductors, Santa Clara, Calif.).

Another feature of the present invention is the use of a secure encrypted communication link between the module 2 and local host 9 and remote host 10 for data security. Any of the various known encryption methods such as CRC encryption and keyed encryption are suitable.

In another embodiment of the invention, the module 2 permits data communication with a mobile telephone in a manner such that a predefined telephone call is placed when the mobile phone is so instructed by the module in order to transmit data via a suitable data protocol as well as a suitable data and transmission backup means.

Another embodiment utilizes data transmission by means of data compression. For example, signals from the individual channels of a multi-channel device such as an EKG sensor array may be compressed via any known data compression means resulting in reduced data transmission time.

Still another embodiment of the present invention is a wireless single channel ECG device. In use, the device consists of one ECG electrode attached to the left arm of a patient and one ECG electrode attached to the right arm of a patient. The ECG electrodes are adhesively attachable thin disks comprising a metal stud to detect the electric potential at the attachment site. The potential differential between the right arm electrode and the left arm electrode generates the signal electrical signal. The stud can be made from any suitable metal however tin is the preferred metal. Suitable electrodes include, but are not limited to, the solid gel electrodes readily available from cardiology equipment suppliers under the trade name 3M REDDOTT™.

The right-arm component contains the electronics for signal amplification, digitization and transmission to a local host via a suitable low power wireless connection. The left-arm component comprises host a power source and the two components are connected by flexible wire circuitry.

The electronics of the ECG device are disposed upon two circular discs that mount directly onto the electrode studs. While the size of such circular disc electrode mounts is not critical, discs in the range of 10 to 40 mm in diameter and 2 to 10 mm in thickness are preferred. The left-arm electrode mount contains a coin cell or similar type battery for power and the right-arm electrode mount contains an amplifier, a micro-controller and a suitable radio frequency transmitter for the wireless transmission of the data to a local host. The left-arm and right-arm mounts are connected via a flexible-circuit cable that contains a conductor to carry the electrical signal from the left-arm electrode to the second component, a conductor to carry electrical power to the second component and a conductor to act as a ground conduit.

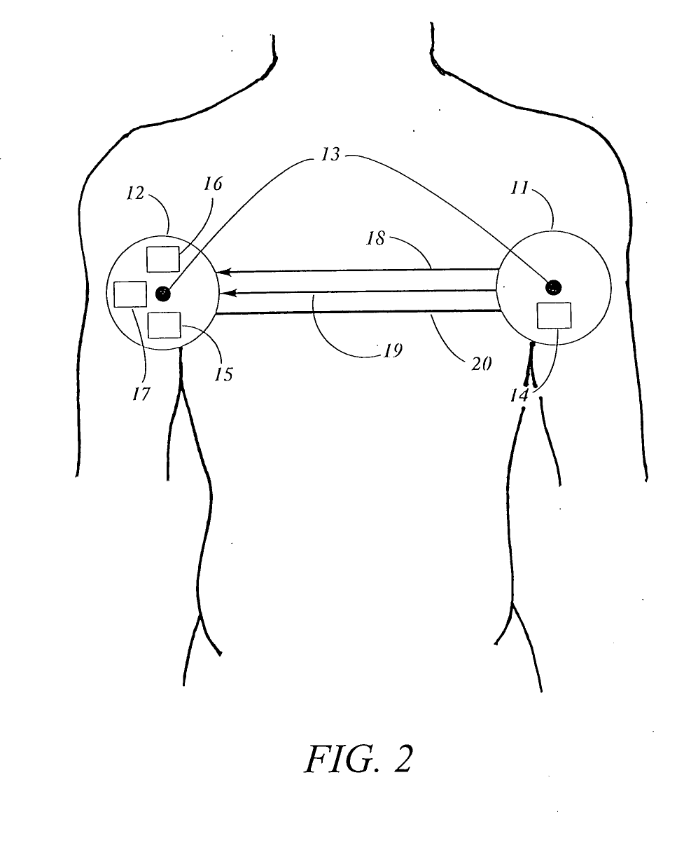

A schematic representation of such a wireless single channel ECG device as mounted on a body is presented in FIG. 2. With reference to FIG. 2, the left arm component 11 and the right arm component 12 of the ECG device are mounted directly onto the electrode studs 13. The device is powered by a coin-type battery 14 contained within the left arm component 11. The right arm component 12 is comprised of a micro-controller 15, an amplifier 16 and a radio frequency transmitter 17. A flexible cable carries the signal 18 from the left-arm electrode, the DC power 19 and the ground conduit 20.

The above description is by way of example only and it will be readily apparent to those of skill in the art that many modifications may be made within the spirit of the invention and that many other embodiments are included as part of this invention.

I claim:

1. A telemetry system for remote patient monitoring comprising one or more biometric sensors capable of being affixed to a patient and wherein said sensors are electronically connected to a module via a first electronic communication link; wherein said module comprises a signal conditioner, an analog to digital converter, a data storage means and an electronic interface capable of establishing and maintaining a second electronic communication link with a local host; and wherein said local host is capable of establishing and maintaining a third electronic communication link with a remote host.

2. The system of claim 1 wherein said data storage means is a random access memory module.

3. The system of claim 1 wherein said data storage means is a removable flash memory card.

4. The system of claim 1 wherein said electronic interface is a wireless interface.

5. The system of claim 1 wherein said wireless interface is powered by a battery.

6. The system of claim 1 wherein said electronic interface is an electrically isolated universal serial bus.

7. The system of claim 1 wherein said local host is a personal digital assistant.

8. The system of claim 1 wherein said local host is a digital computer.

9. The system of claim 1 wherein said one or more sensors is an EKG sensor array.

10. The system of claim 9 wherein said EKG sensor array is a 12 lead EKG sensor array.

11. The system of claim 9 wherein said EKG sensor array is a 3 lead EKG sensor array.

12. The system of claim 1 wherein said one or more sensors is a blood oxygen sensor.

13. The system of claim 1 wherein said one or more sensors is a temperature sensor.

14. The system of claim 1 wherein said one or more sensors is a medical imaging sensor.

15. The system of claim 1 wherein said one or more sensors is a blood glucose level sensor.

16. The system of claim 1 wherein the electronic communication link between the self-contained module and local host is a secure encrypted link.

17. The system of claim 1 wherein the electronic communication link between the local host and the remote host is a secure encrypted link.

18. A single-channel wireless ECG system for remote patient monitoring comprising a first component attachable to the left arm of a patient and a second component attachable to the right arm of a patient wherein said first component comprises a left-arm ECG electrode and a power source; said second component comprises a right-arm ECG electrode, a micro-controller, an amplifier and a radio frequency transmitter; and wherein said first component and said second component are joined by a flexible cable comprising a conductor to carry an electrical signal from said left-arm electrode to said second component, a conductor to carry electrical power to said second component and a conductor to act as a ground conduit.

19. A method for remote patient monitoring comprising the steps of:
providing one or more biometric sensors affixed to the patient and connected to a module via a first electronic communication link; wherein said module comprises a signal conditioner, an analog to digital signal converter, a digital data storage means and an electronic interface capable of establishing and maintaining a second electronic communication link with a local host; wherein said local host is capable of establishing and maintaining a third electronic communication link with a remote host;
transmitting electronic signals from said biometric sensors to said module via said first electronic communication link;
converting the electronic signals within said module to digital data;
transmitting said digital data from said module to said local host via said second electronic communication link; and
transmitting said digital data from said local host to said remote host via said third electronic communication link.

20. The method of claim 19 further comprising the step of storing said digital data on the digital data storage means within said module while simultaneously transmitting said digital data to said local host.

21. The method of claim 19 further comprising the step of storing said digital data on the digital data storage means within said module prior to the step of transmitting said digital data to said local host via said second electronic communication link.

22. The method of claim 19 wherein said first electronic communications link is a wired communications link.

23. The method of claim 19 wherein said first electronic communications link is a wireless communications link.

24. The method of claim 19 wherein said second electronic communications link is a wired communications link.

25. The method of claim 19 wherein said second electronic communications link is a wireless communications link.
